(19) United States
(12) Patent Application Publication
(10) Pub. No.: US 2006/0046875 A1
(43) Pub. Date: Mar. 2, 2006

(54) American football defensive player trainer

(76) Inventor: Charles P. Forrest SR., Stapleton, AL (US)

Correspondence Address: H. Jay Spiegel, P.O. Box 11, Mount Vernon, VA, 22121, US

(21) Appl. No.: 10/924,801

(22) Filed: Aug. 25, 2004

(51) Int. Cl. A61F 9/00

(52) U.S. Cl. 473/441

(57) ABSTRACT

A frame is mounted on wheels for easy transport of the device from place to place on a practice field. The frame has three pads in a line simulating offensive interior linemen. When the right-hand pad is struck by a player and reciprocated backward, a mechanism causes a latch to be tripped that results in a further pad behind the right-hand pad pivoting under spring bias to a position lateral of and slightly behind the right-hand pad where the further pad may be struck by the player. When the further pad is struck by the player, it is pushed back into a cocked position where it remains until the right-hand pad is again struck from the front. When the left-hand pad is struck from the front, the same result occurs symmetrically. On the rear of the device, two to three additional pads are provided that depend downwardly from inverted L-shaped arms pivotably mounted on the frame of the inventive device.

### BACKGROUND OF THE INVENTION

The present invention relates to an American football defensive player trainer. In the prior art, numerous examples exist of training devices for players playing the American-style game of football. Numerous examples of blocking and tackling dummies, sleds and other devices exist. However, up until now, there has never been any device devised that accurately simulates the scenario that confronts a defensive player every time the ball is snapped.

In the real world of American football, a defensive player such as a lineman or linebacker confronts large offensive linemen who attempt to block the defensive player to permit running and passing plays to succeed. When the defensive player defeats the blocker, he is often confronted with another blocker who stands between him and a running back, receiver or quarterback.

Numerous devices exist that permit a defensive player to practice defeating the block of an offensive player. However, up until now, no device has been produced that permits the defensive player to practice the scenario in which a first offensive player is confronted and when that blocker is defeated, a second blocker comes into play. It is with this thought in mind that the present invention was developed.

The following prior art is known to Applicant:

| | |
|---|---|
| 704,265 to McMaster | 4,186,922 to Ketchum |
| 1,176,730 to Ashton | 4,447,056 to Dalton |
| 2,620,188 to Malagio | 4,477,076 to Monaco |
| 3,062,547 to Kopp | 6,261,195 to Shingleton |
| 3,390,880 to Forrest | 6,685,581 to Krause et al. |
| 3,658,332 to Adduci et al. | |

The above-listed U.S. Patents teach a variety of examples of blocking and tackling dummies and sleds. The present invention differs from the teachings of these patents as contemplating a defensive player trainer in which a first dummy is hit and causes actuation of a mechanism causing a second dummy to be moved to a blocking position that must be defeated by the player who struck the first dummy.

### SUMMARY OF THE INVENTION

The present invention relates to an American football defensive player trainer. The present invention includes the following interrelated objects, aspects and features:

(1) In the preferred embodiment of the present invention, a frame is provided that supports all of the pads of the present invention and may include a seat for the operator who typically is a football coach. The frame may be mounted on wheels for easy transport of the device from place to place on a practice field.

(2) In a first aspect, the frame has mounted thereon, in the preferred embodiment, three pads in a line simulating offensive interior linemen. The centrally located pad may be mounted on a support allowing it to reciprocate forward and backward responsive to being struck by a player. The left-hand and right-hand pads also reciprocate. However, when they reciprocate, other functions of the inventive device result.

(3) When the right-hand pad is struck by a player and reciprocated backward, a linkage mechanism attached thereto including an elongated arm causes a latch on another arm to be tripped that results in a further pad behind the right-hand pad pivoting under spring bias to a position lateral of and slightly behind the right-hand pad where the further pad may be struck by the player. When the further pad is struck by the player, it is pushed back into a cocked position where it remains until the right-hand pad is again struck from the front.

(4) Similarly, when the left-hand pad is struck from the front, a linkage mechanism attached thereto including an elongated arm, and symmetrical with the linkage mechanism of the right-hand pad, trips a latch on another arm that causes another further pad behind the left-hand pad to swing outwardly to a position lateral of and slightly behind the left-hand pad where this further pad may be struck by the player. In similar fashion to the first-mentioned further pad, when the further pad behind the left-hand pad is struck, it is moved to a latched position where it remains until the left-hand pad is again struck.

(5) On the rear of the inventive device, two to three additional pads are provided which preferably depend downwardly from inverted L-shaped arms that are pivotably mounted on the frame of the inventive device. One such pad is mounted on such a support that causes the pad to hang slightly laterally of and behind the further pad behind the right-hand pad in a position where the further pad behind the right-hand pad has been deployed. One or more tension springs attached between the support and the frame retain the hanging pad in its position until it is grasped by the defensive player, whereupon the support and pad may be swung rearwardly to a position directly behind the frame. In the preferred embodiment, a limit stop limits the degree of pivoting of the support so that the hanging pad may only be pivoted a prescribed distance behind the frame.

(6) In similar fashion, a further support is provided in the left rear of the frame that supports a hanging pad in an initial position as held by spring bias slightly rearward and laterally of the further pad located behind and laterally of the left-hand pad when the further pad is deployed. The hanging pad on the left side of the device operates in analogous fashion to that of the hanging pad on the right-hand side of the device including the preferred provision of a limit stop to limit the degree by which the hanging pad may be swung on its support rearward of the device.

(7) If desired, a third inverted L-shaped support may be provided on the center of the rear edge of the frame to support a third hanging pad that may be swung leftward and rightward by a player.

As such, it is a first object of the present invention to provide an American football defensive player trainer.

It is a further object of the present invention to provide such a device in which left-hand and right-hand pads may be struck by a player from the front to cause respective further pads to swing into a position where they must be evaded by the defensive player.

It is a still further object of the present invention to provide such a device in which additional pads are mounted on supports on the rear of the frame of the inventive device and may be swung when grasped by a player.

It is a still further object of the present invention to provide such a device in which the further pads behind the left-hand and right-hand pads may be swung back into positions where they are locked until the left-hand and right-hand pads are again struck, respectively.

It is a still further object of the present invention to provide such a device in which the frame is mounted on wheels allowing easy transport from location to location.

These and other objects, aspects and features of the present invention will be better understood from the following detailed description of the preferred embodiment when read in conjunction with the appended drawing figures.

### BRIEF DESCRIPTION OF THE DRAWINGS

FIG. 1 shows a front perspective view of the present invention.

FIG. 2 shows a rear perspective view of the present invention.

FIG. 3 shows a right side rear perspective view of the present invention.

FIG. 4 shows a top view of a mechanism interconnected between a right-hand pad and a further pad behind the right-hand pad in a position in which the further pad has been activated to pivot laterally and behind the right-hand pad.

FIG. 5 shows a top view of the mechanism shown in FIG. 4, but with the mechanism in the cocked position and the further pad located directly behind the right-hand pad.

FIG. 6 shows a side perspective view of the mechanism shown in FIGS. 4 and 5 in the cocked position.

FIG. 7 shows a rear right-side perspective view of the same mechanism shown in FIG. 6 in the cocked position.

FIG. 8 shows the same mechanism illustrated in FIGS. 6 and 7, but from a front right-side perspective and in a position where the mechanism has been activated to pivot the further pad laterally and rearward of the right-hand pad.

FIG. 9 shows a perspective view of the orientation of parts of FIG. 8, but from the front left-side thereof.

FIG. 10 shows a view of the same parts looking from the rear and with the device cocked as shown in FIGS. 6 and 7.

FIG. 11 shows a perspective view from above of the further pad behind the right-hand pad along with its support arm and actuating mechanism.

FIG. 12 shows a view similar to that of FIG. 11, but with the pad activated and pivoted to its extended position.

FIG. 13 shows a close-up rear view of the right-hand pad and a portion of the actuating mechanism for the further pad associated therewith and in the cocked position.

FIG. 14 shows a perspective view from the left rear of the further pad behind the left-hand pad along with its support and the left-hand pad as well as the actuating mechanism in the cocked position.

FIG. 15 shows a view similar to that of FIG. 14, but with the further pad in the actuated extended position.

FIG. 16 shows a view looking from the front of the device of the rear right-hand hanging pad and the distal end of its support.

FIG. 17 shows the proximal end of the support for the left rear hanging pad showing the details of support and limit stops.

FIG. 18 shows a close-up perspective view of the proximal end of the support for the rear right-hand pad.

FIG. 19 shows a rear perspective view of an indicator mechanism of the present invention in its retracted uplifted position.

FIG. 20 shows the indicator mechanism of FIG. 19 in its forwardly extended downwardly pivoted position.

FIG. 21 shows a front view of the indicator mechanism in the position shown in FIG. 20.

FIG. 22 shows the indicator mechanism pivoted to the left-hand side.

### SPECIFIC DESCRIPTION OF THE PREFERRED EMBODIMENT

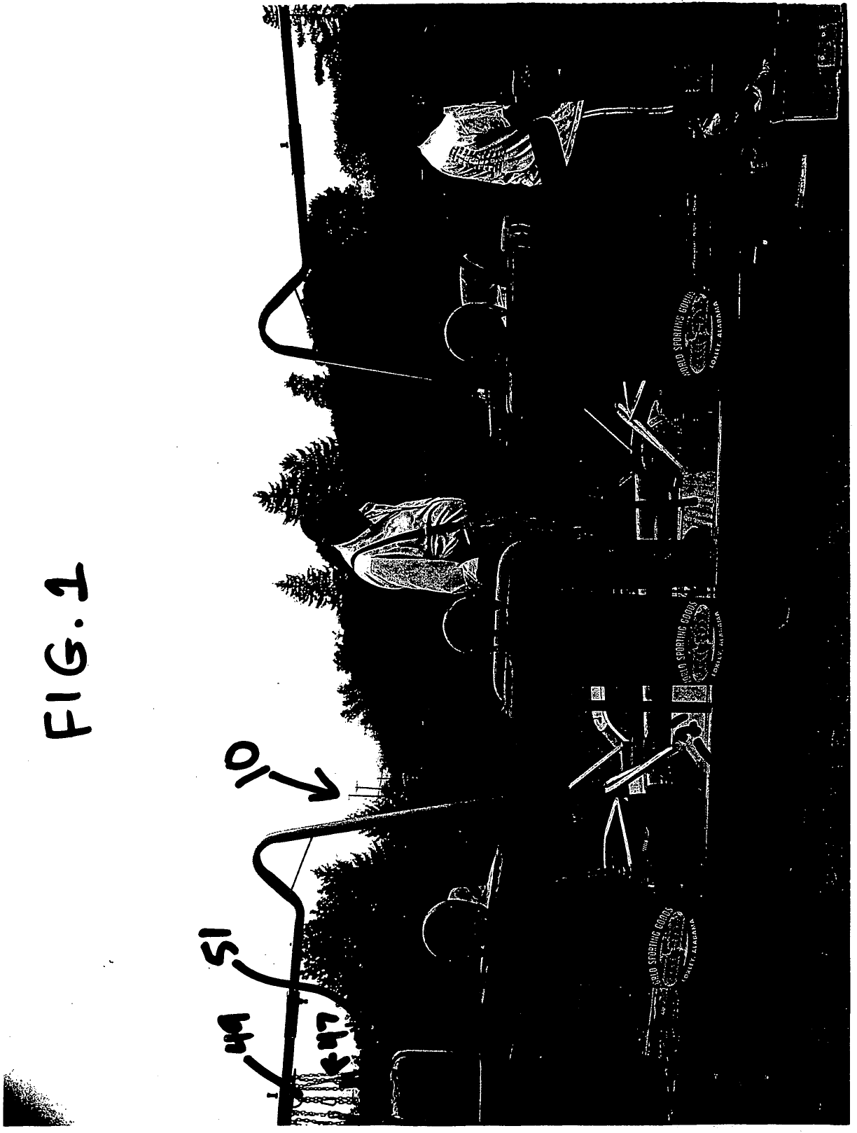

With reference first to FIGS. 1-3, the present invention is generally designated by the reference numeral 10. As seen in these figures, the inventive device includes a frame 11 including a plate 13 on which a user may stand (FIG. 1) and from which the various aspects of the present invention emanate.

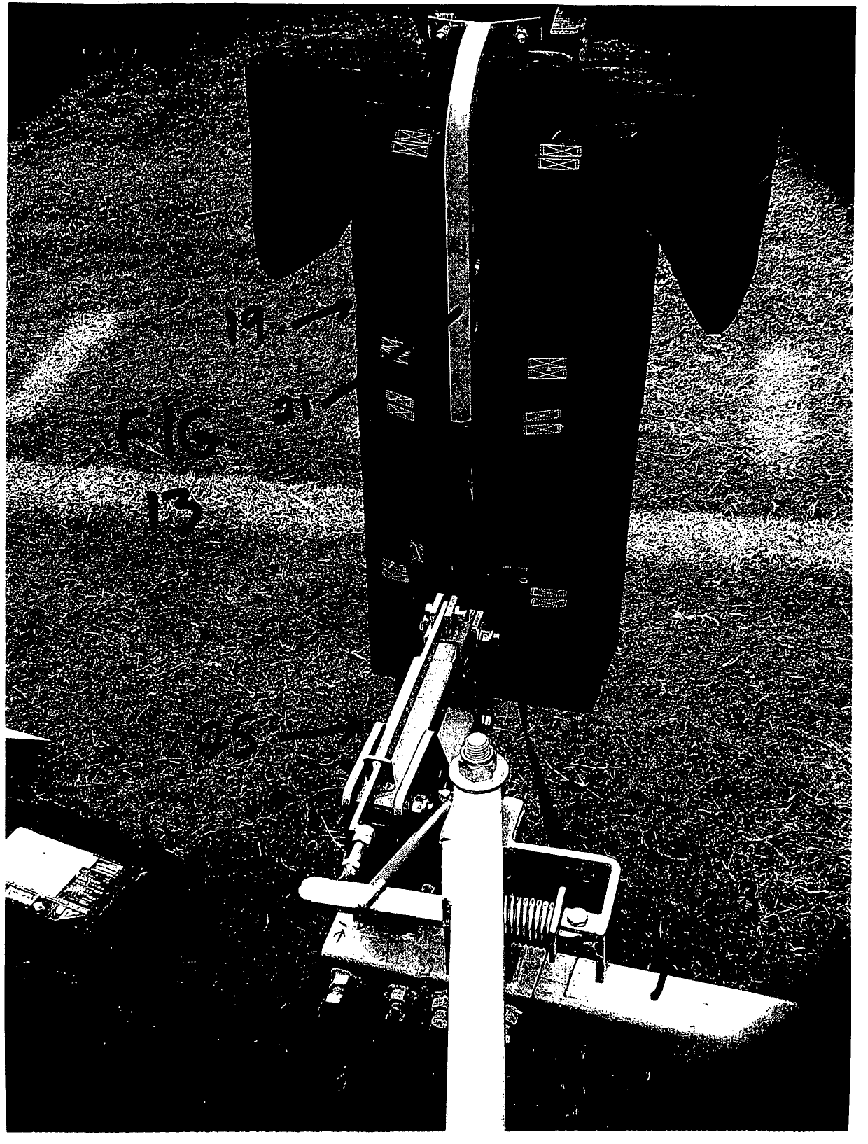

FIG. 1 shows the inventive device 10 from the front and shows pads 15, 17 and 19 mounted on the front edge 21 of the frame. FIG. 13 shows the rear of the pad 19 and depicts the mounting means for the pad 19 including a support consisting of an upwardly extending bar 21 fixed to a horizontal tube 23 via an actuating mechanism 25 that will be described in greater detail hereinafter. The bar 23 includes the front edge 21 best seen in FIG. 1.

With reference back to FIGS. 1-3, it is seen that behind the right-hand pad 19, a further pad 27 is provided. Similarly, behind the left-hand pad 15, a further pad 29 is provided. The details of these pads will be described in further detail hereinafter.

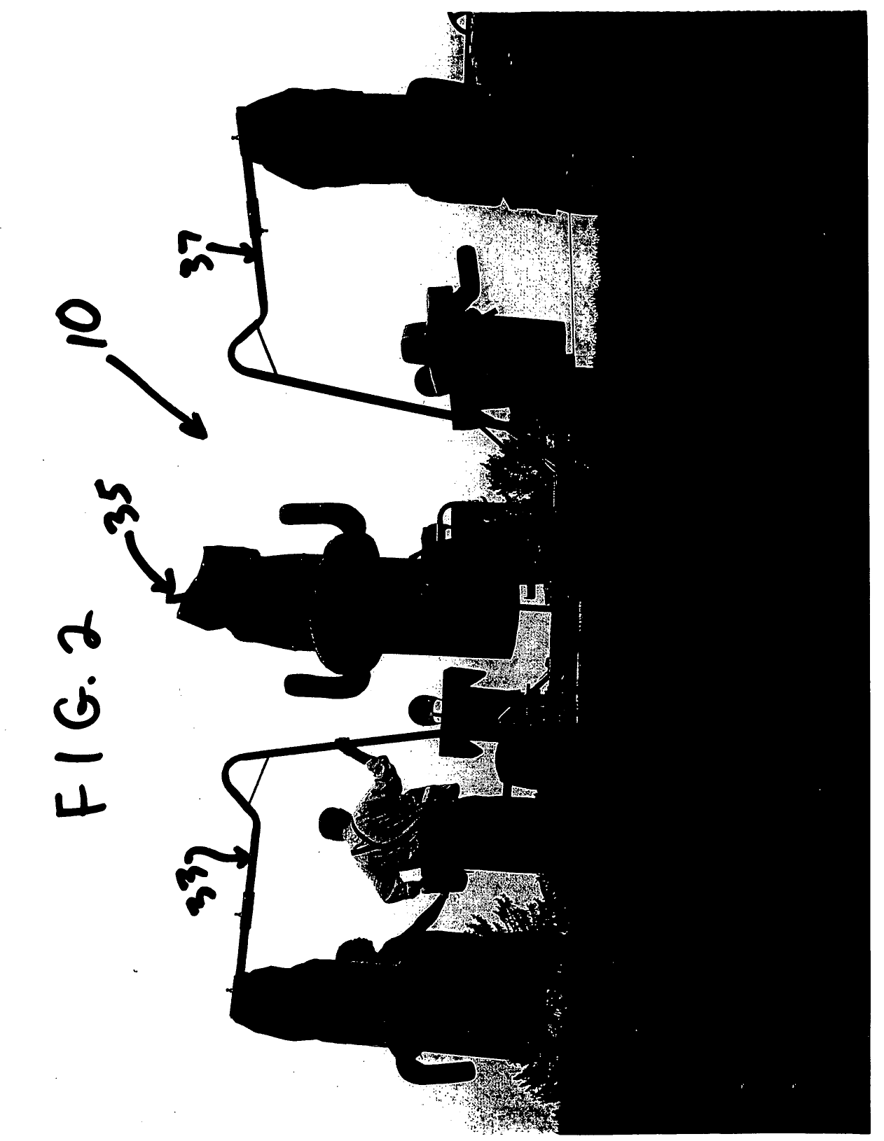

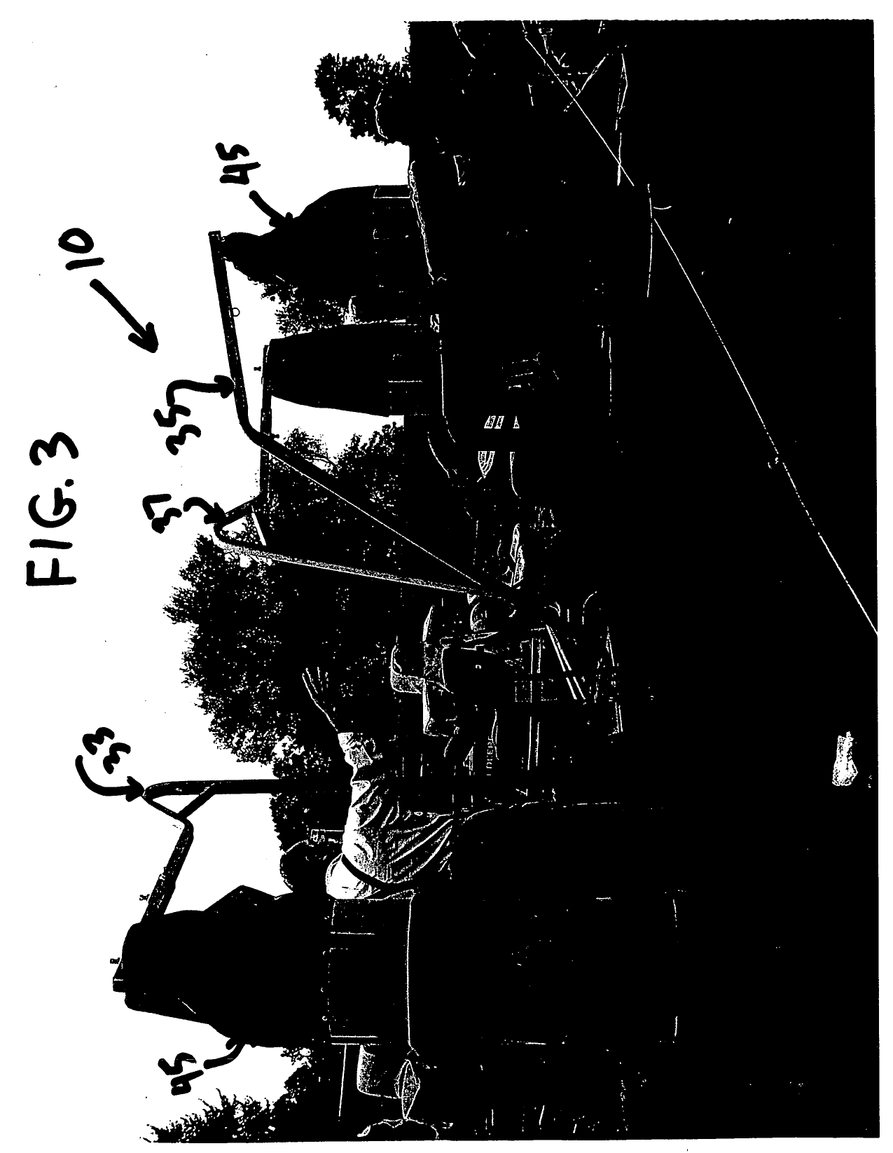

With more particular reference to FIGS. 2 and 3, the rear edge 31 of the frame 11 includes mounts for three inverted L-shaped supports designated by the reference numerals 33, 35 and 37. With particular reference to FIGS. 2 and 3, the support 33 supports a hanging pad 39 that is supported generally behind the further pad 27. Similarly, the support 37 supports a hanging pad 41 that is located generally behind the further pad 29. The support 35 is centrally located along the rear edge 31 of the frame 11 and supports a further hanging pad 43 that may pivot side-to-side about the rear edge 31 of the frame 11. The pads 39, 41 and 43 may be hung from their respective supports 33, 35 and 37 using any suitable means.

In FIGS. 2 and 3, the pads 39, 41 and 43 are shown with water-repellent boots 45 mounted over their top surfaces. These boots obscure the manner of mounting of the pads to their respective supports. However, in FIG. 1, the pad 39 is seen without its boot 45 and makes clear that one way the pad 39 and the other pads 41 and 43 may be supported from their respective supports is through the use of chains 47 suspended from eyes 49 on the supports, respectively, and straps 51 on the upper surface of the pads to hook with the chains 47. This is but one example of a way by which the pads 39, 41 and 43 may be suspended from their respective supports 33, 35 and 37.

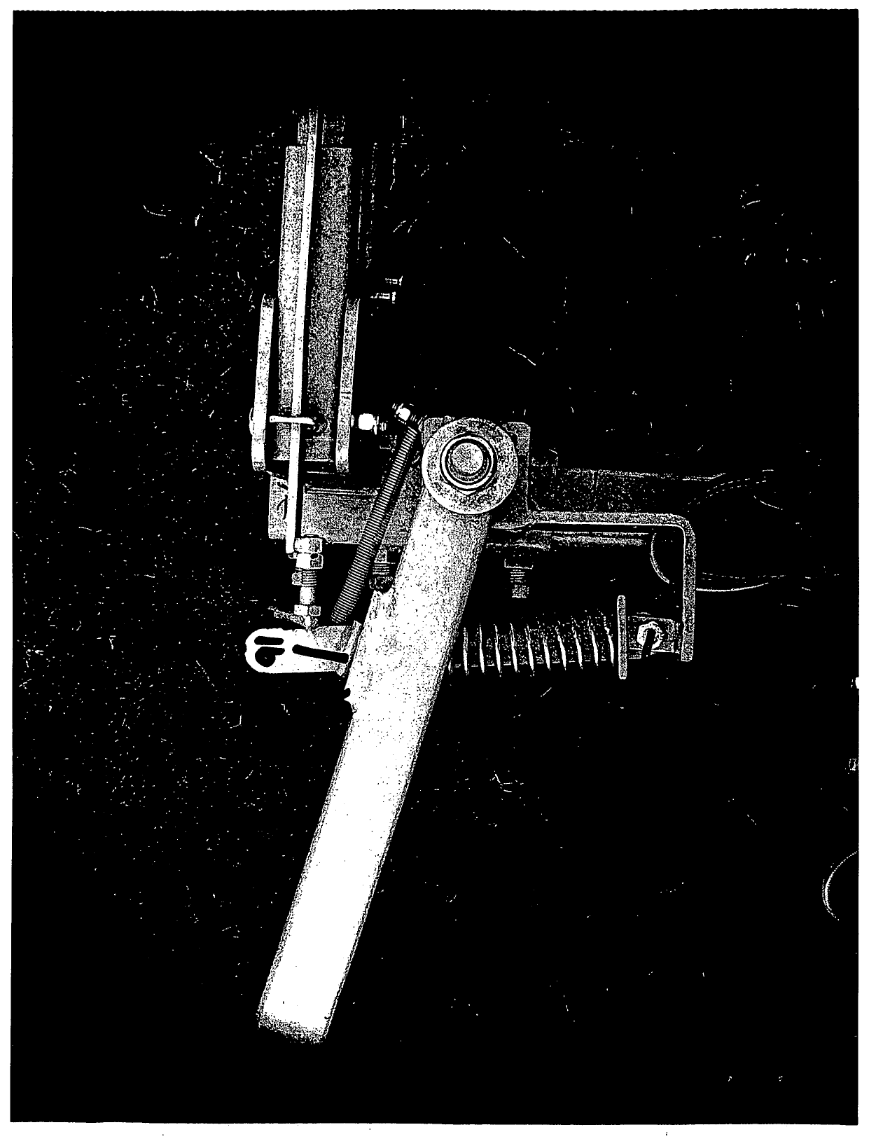

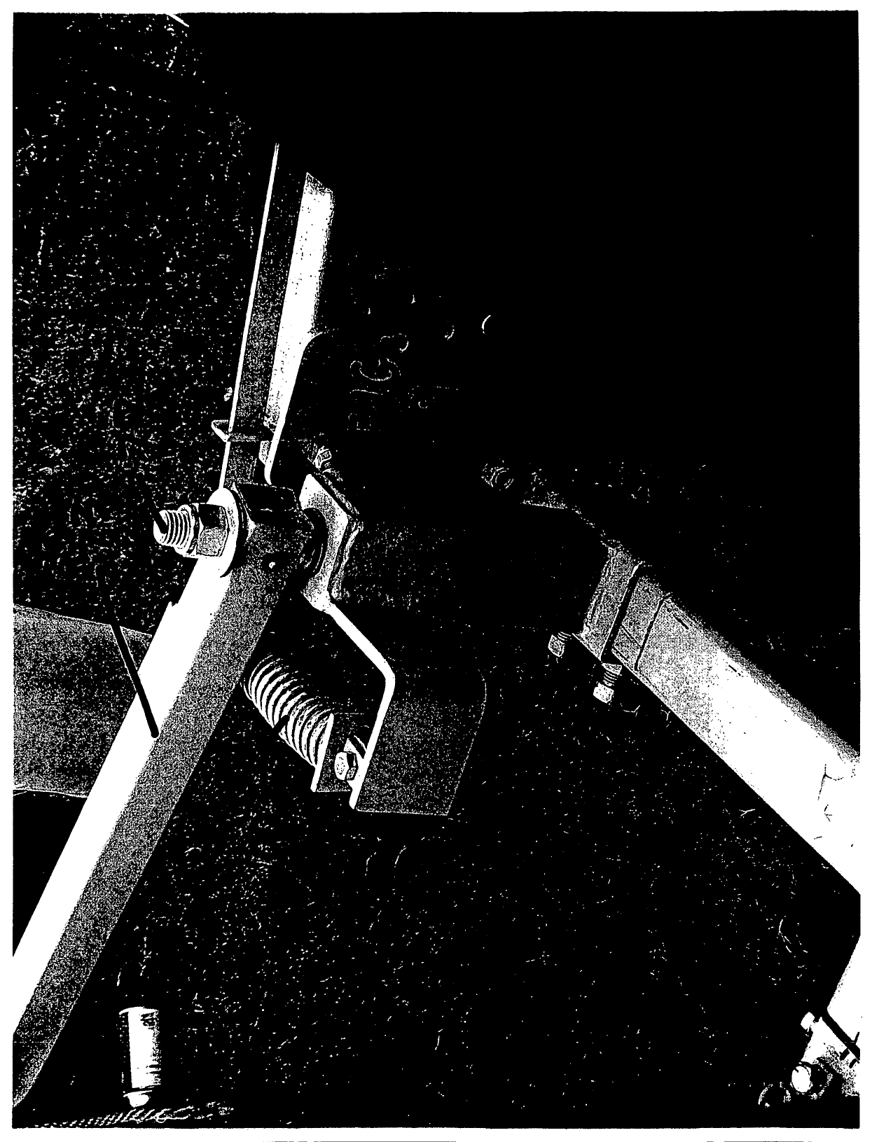

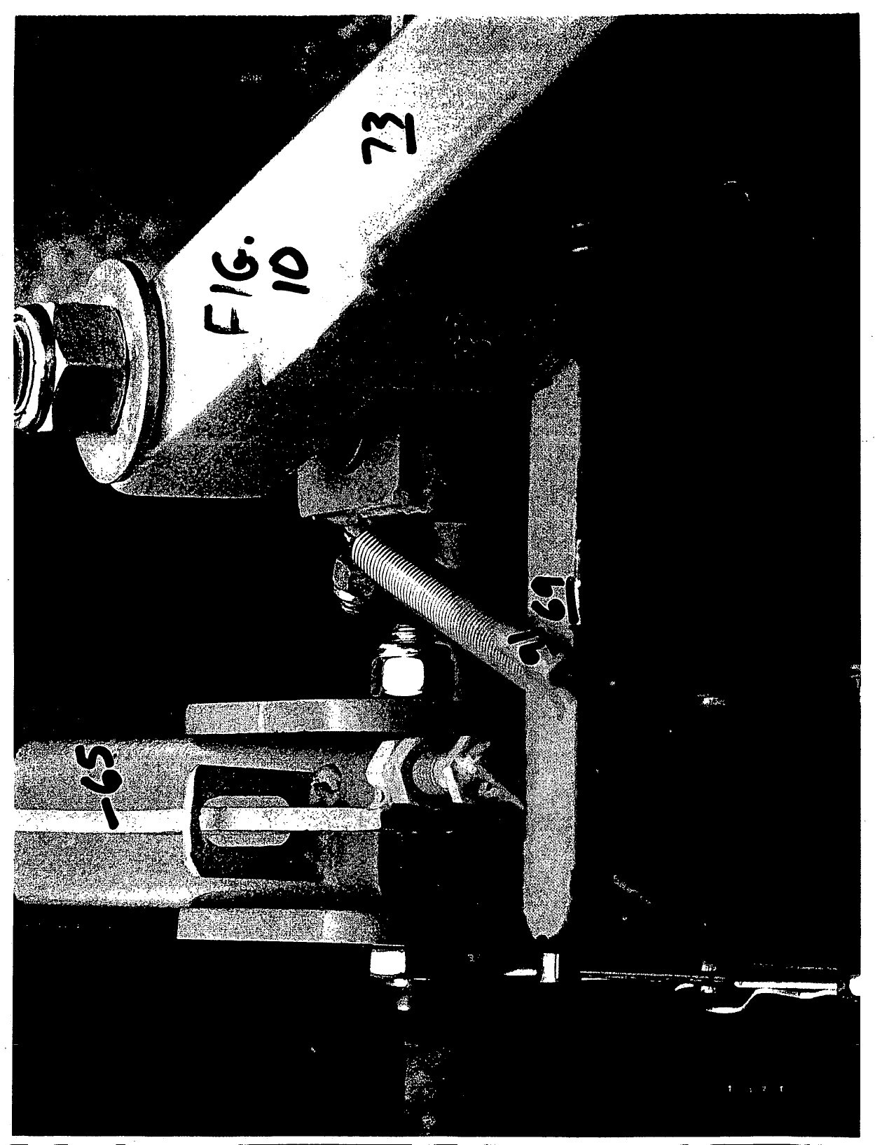

With reference, now, to FIGS. 4-15, the preferred manner of operation of the front pads 15 and 19 and the further pads 27 and 29 will now be explained.

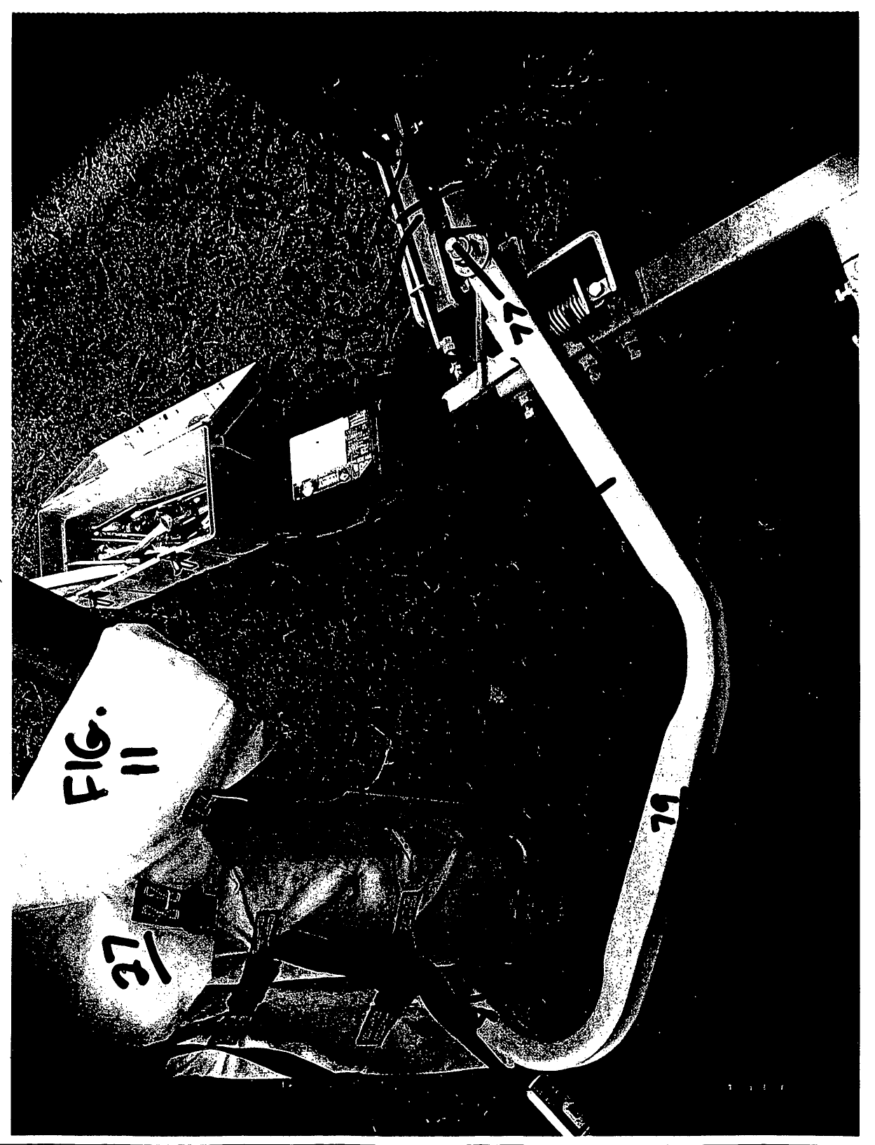

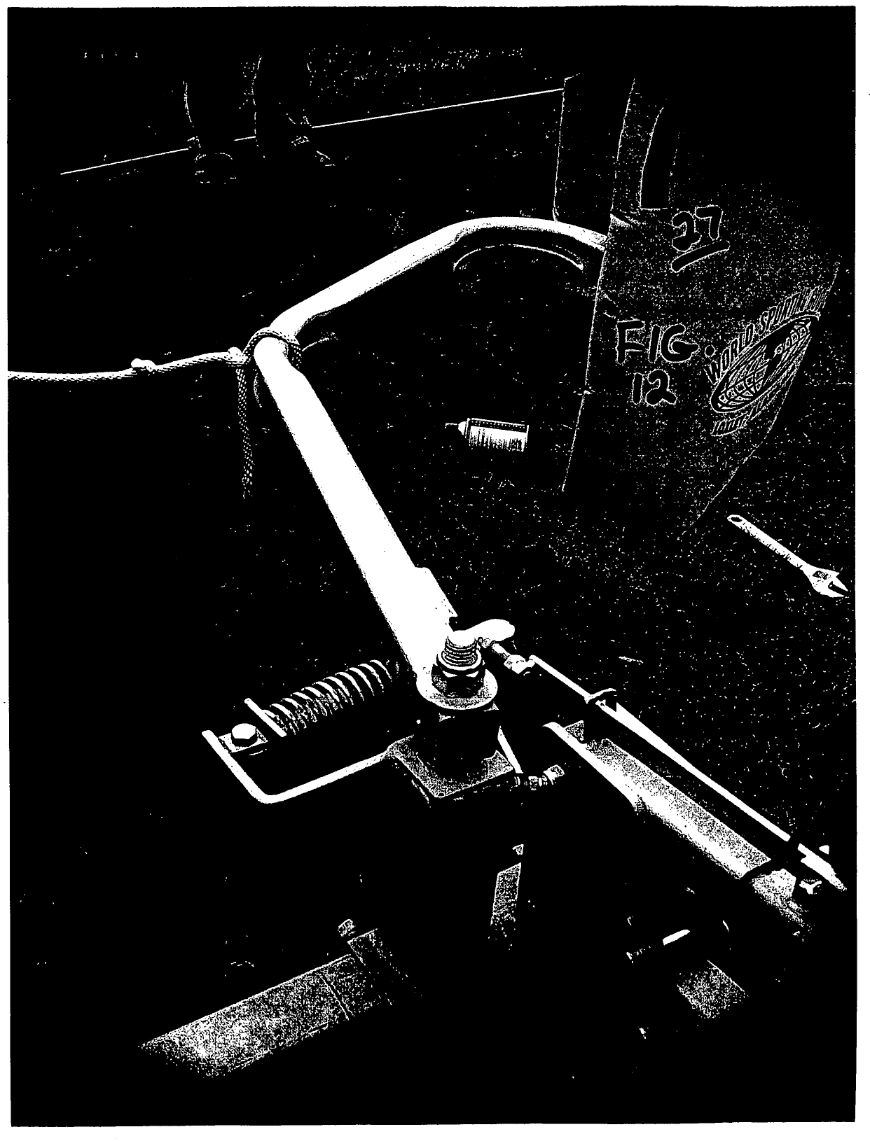

Reference is first made to FIGS. 11 and 12 which show the interconnection or linkage between the pads 19 and 27. The pad 19 is mounted on the support 21 which includes a horizontal portion 61 that telescopes over a horizontal tube 63 permitting the pad 19 to be struck and moved rearward in the view of FIG. 1.

An actuating arm 65 is also attached to the support 21 for the pad 19 and moves in the rearward direction in the view of FIG. 1 when the pad 19 is struck from the front. The end of the arm 65 remote from the pad 19 is designated by the reference numeral 67, and engages the end 71 of the release arm 69 as seen in particular in FIG. 11. As also seen in FIGS. 11 and 12, the pad 27 is supported on an elongated arm 73 that includes a proximal portion 75 attached to the frame 11 at a pivot 77. The proximal portion 75 is connected to an angularly connected section 79 that in turn has a curved end 81 attached to a vertical support similar to the support 21 to support the pad 27 as shown.

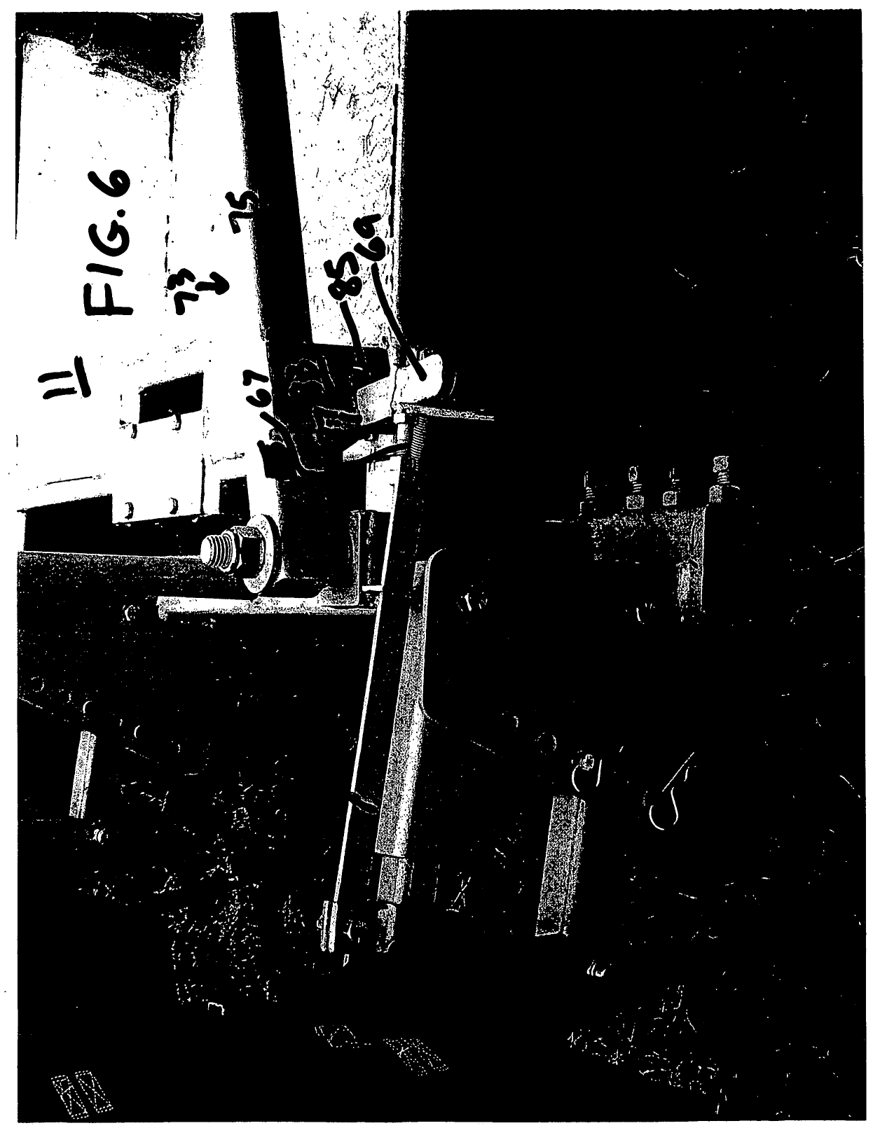

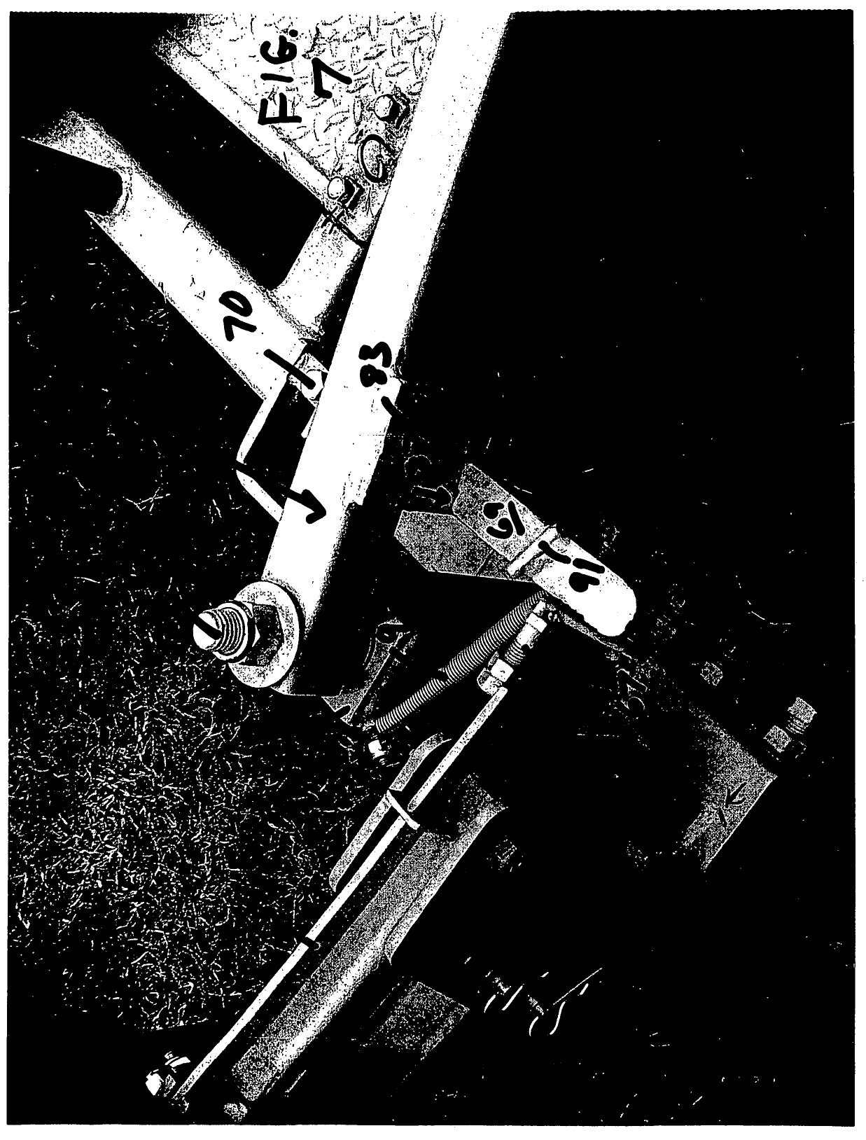

As seen in FIGS. 6 and 7, the proximal portion 75 of the arm 73 has a downwardly depending rectangular plate 83 with a slot 85 therethrough that receives the arm 69 therethrough. As seen in FIGS. 6 and 7, the arm 69 has a notch 87 that engages the side of the slot 85 when the arm 73 is in the cocked position, and locks the arm in that position despite the spring force of the compression spring 90 (FIG. 11) which in that position tends to bias the arm 73 to the position of outward pivoting of the further pad 27.

The arm 69 also includes a bar 91 on its top surface that provides a limit stop engageable by the plate 83 when the pad 27 has been pivoted to the position shown in FIG. 12. FIG. 4 shows the plate 83 engaging the limit stop 91 in the extended position of the pad 27.

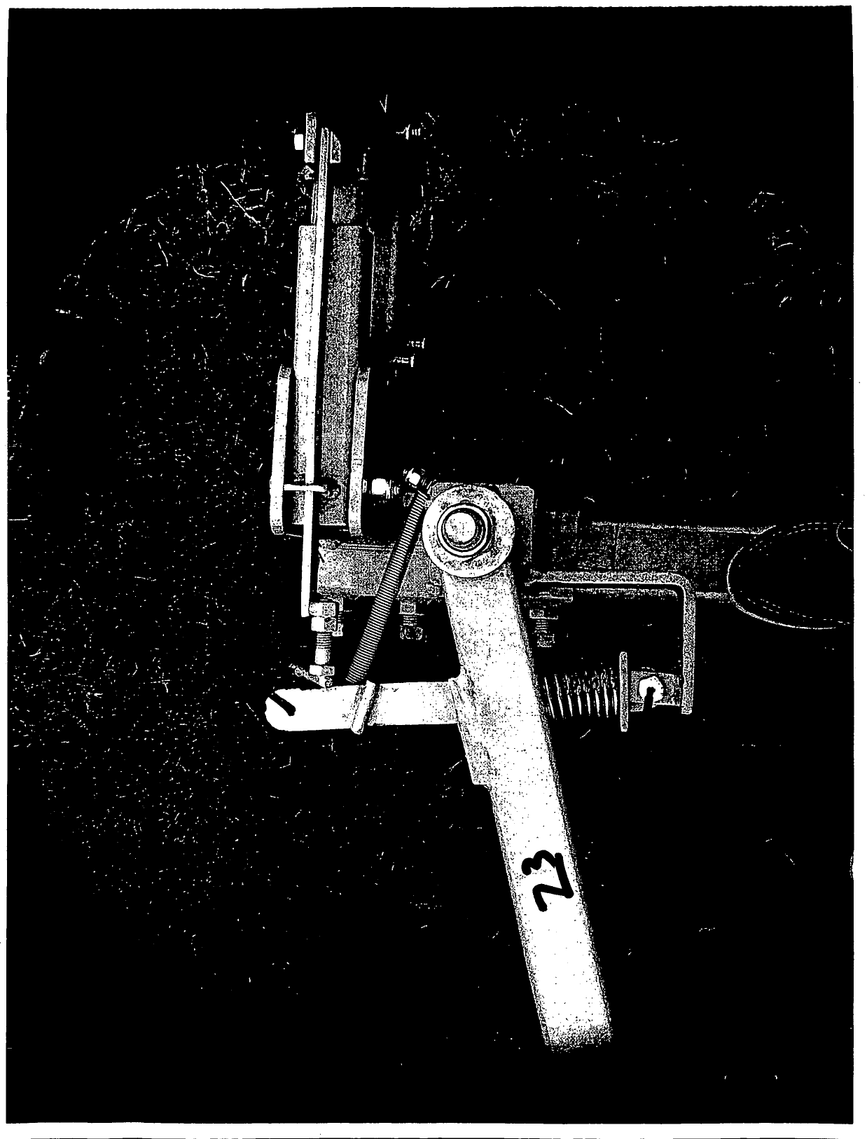

With reference to FIG. 7, a tension spring 95 is attached between a stub 97 supporting the pivot 77 of the arm 73 and the arm 69 to tend to pivot the arm 69 about its pivot 70 (FIGS. 5 and 6) in the clockwise direction in the view of FIGS. 4 and 5, tending to pivot the arm 69 to a position in which the notch 87 in the arm 69 engages the side edge of the slot 85 to lock the arm 73 in the cocked position seen, for example, in FIG. 11.

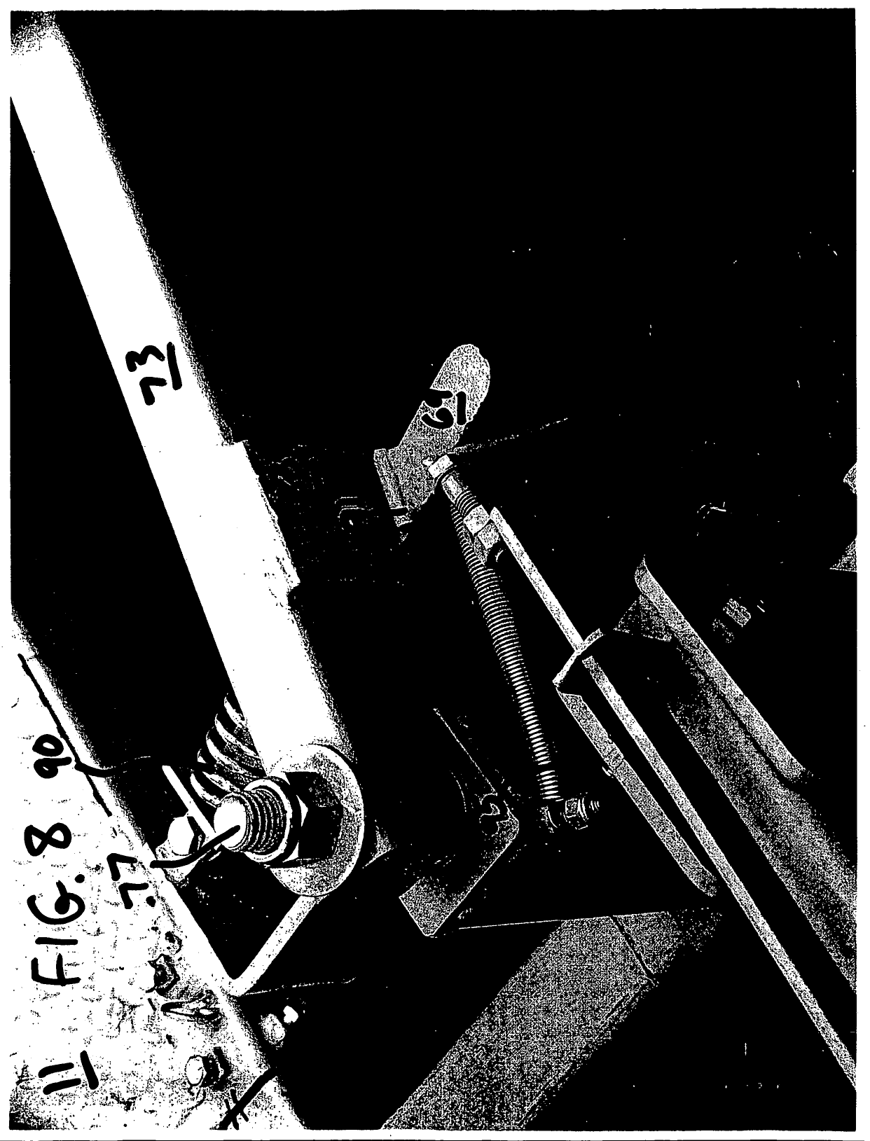

As should now be understood, when the pad 19 is pushed rearwardly in the orientation shown in FIG. 1, such action causes rearward movement of the pad 19 and rearward reciprocation of the arm 65. Thus, the distal end 67 of the arm 65 engages the end 71 of the arm 69 (FIGS. 6 and 7) causing the arm 69 to pivot counterclockwise in the orientation shown in FIG. 5. When the notch 87 (FIGS. 6 and 7) is released from the edge of the notch 85, under the force of the spring 90, the arm 73 is pivoted to the position shown in FIGS. 4 and 12 so that the further pad 27 is extended laterally and forwardly to a position where it is slightly rearward and to the side of the pad 19. This position is also seen in FIG. 8 which also shows the limit stop 91 engaged by the plate 83 to stop further pivoting movement of the arm 73 with the further pad 27 attached thereto. In the position shown, in particular, in FIG. 8, the spring 95 is under tension tending to want to pivot the arm 69 to the position shown in FIG. 5 in which the notch 87 engages the side of the slot 85 in the plate 83 to cock the arm 73. However, in the position shown in FIG. 8, for example, as well as FIG. 4, the arm 73 is held in its largest position of extension by the force of the spring 90 and engagement of the plate 83 with the limit stop 91. Thus, when a player strikes the further pad 27, after striking the pad 19, such force imposed on the further pad 27 causes the arm 73 to move in the counterclockwise direction in the view of FIGS. 4 and 5, from the position shown in FIG. 4 to the position shown in FIG. 5 in which the arm 73 is re-cocked in the position shown in FIG. 5 and locked in that position through interaction of the notch 87 and the slot 85.

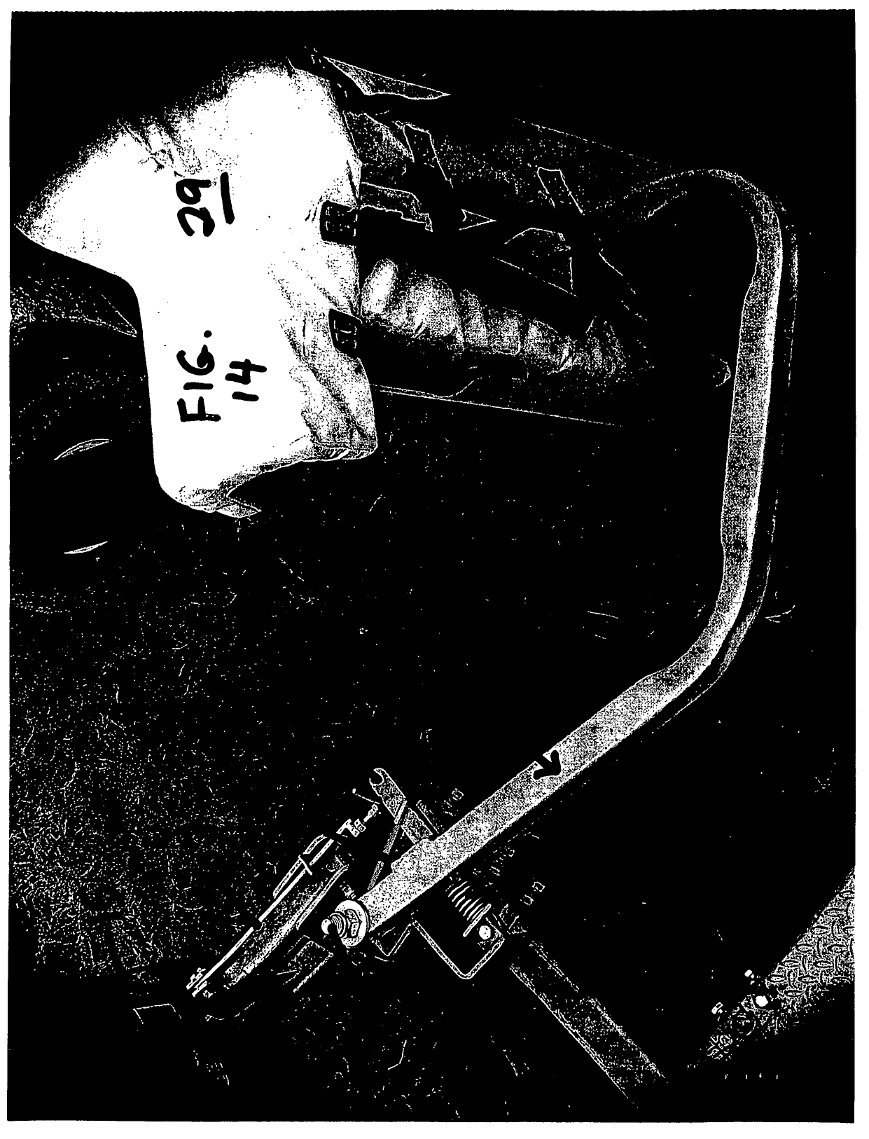

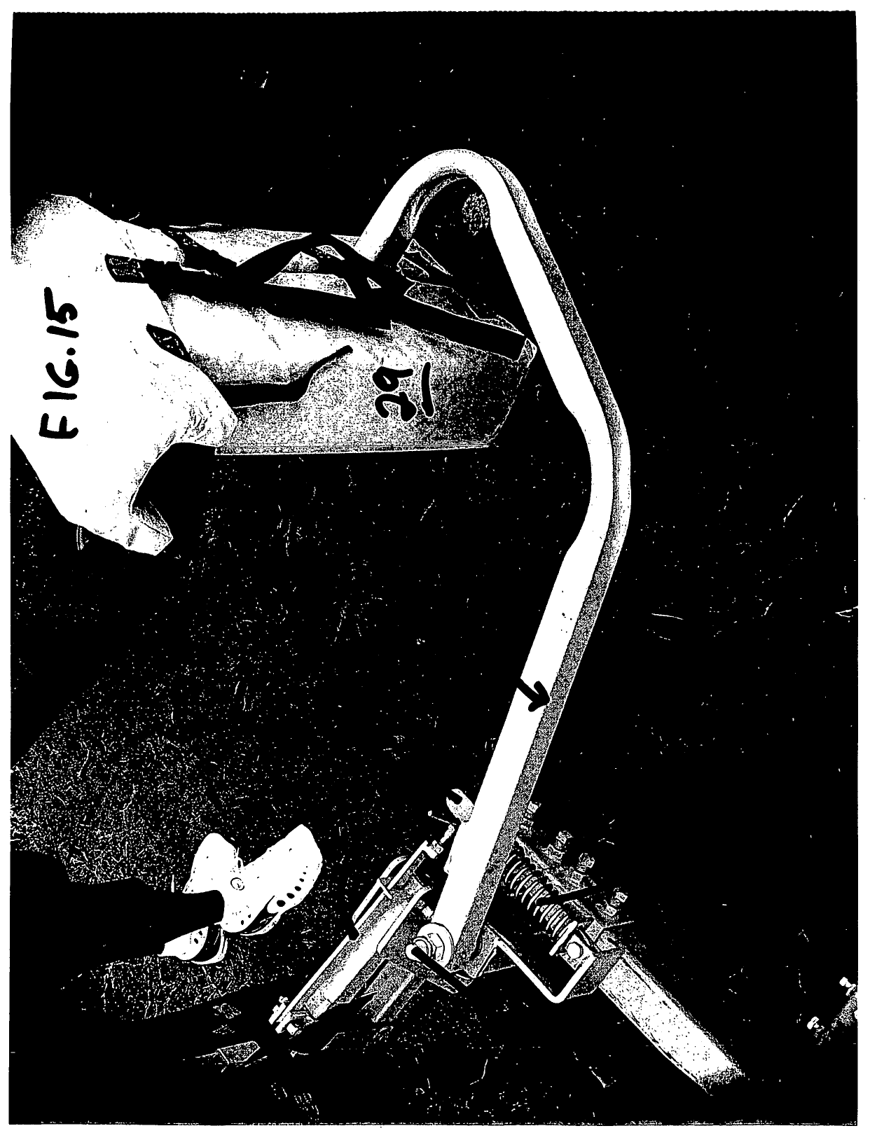

Operation of the further pad 29 located behind the left-hand pad 15 is the same as the operation described above concerning the pads 19 and 27, but symmetrical with respect thereto. In this regard, FIGS. 14 and 15 show the pads 15 and 29 and the associated structure including the arm 101, the arm 103, the spring 105, the actuator arm 107, the pivot 109, and the spring 111, among other structures. It should be well understood by those of ordinary skill in the art that the components associated with the pads 19 and 27 correspond with the components associated with the pads 15 and 29, based upon the description set forth hereinabove.

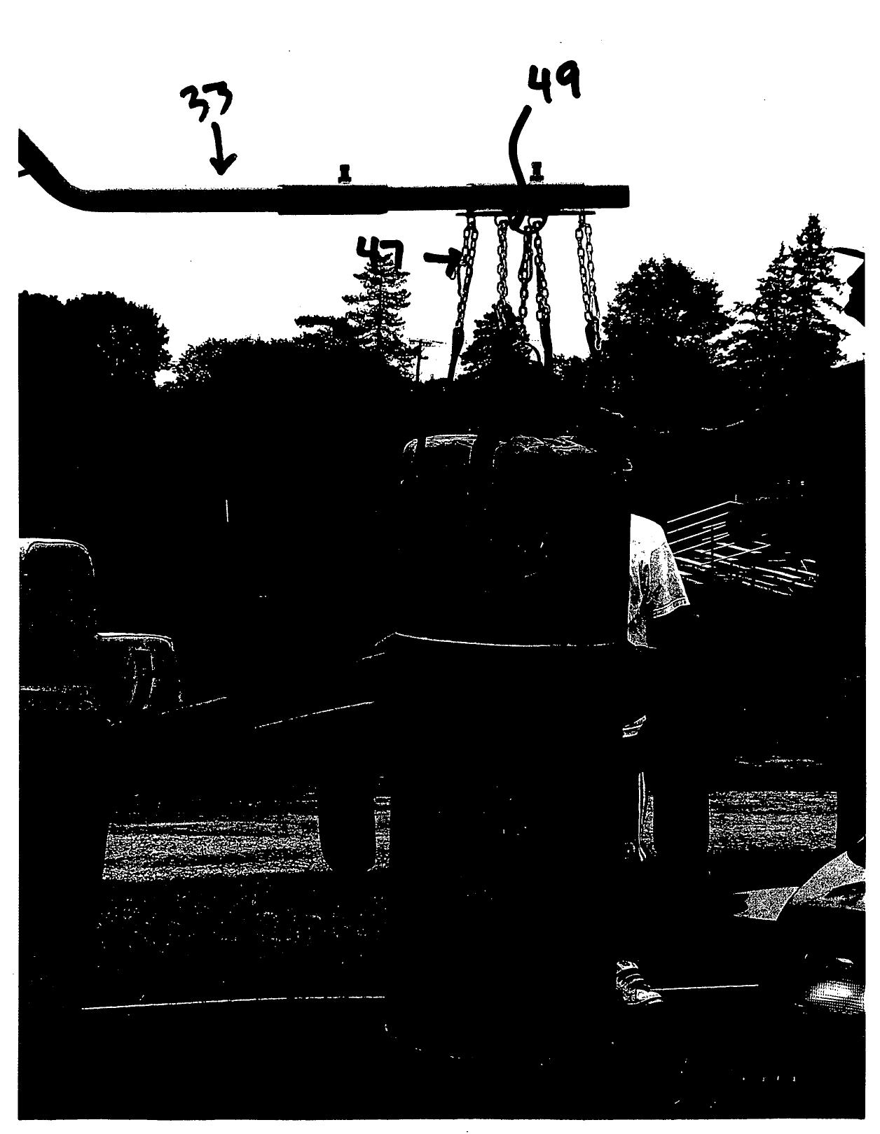

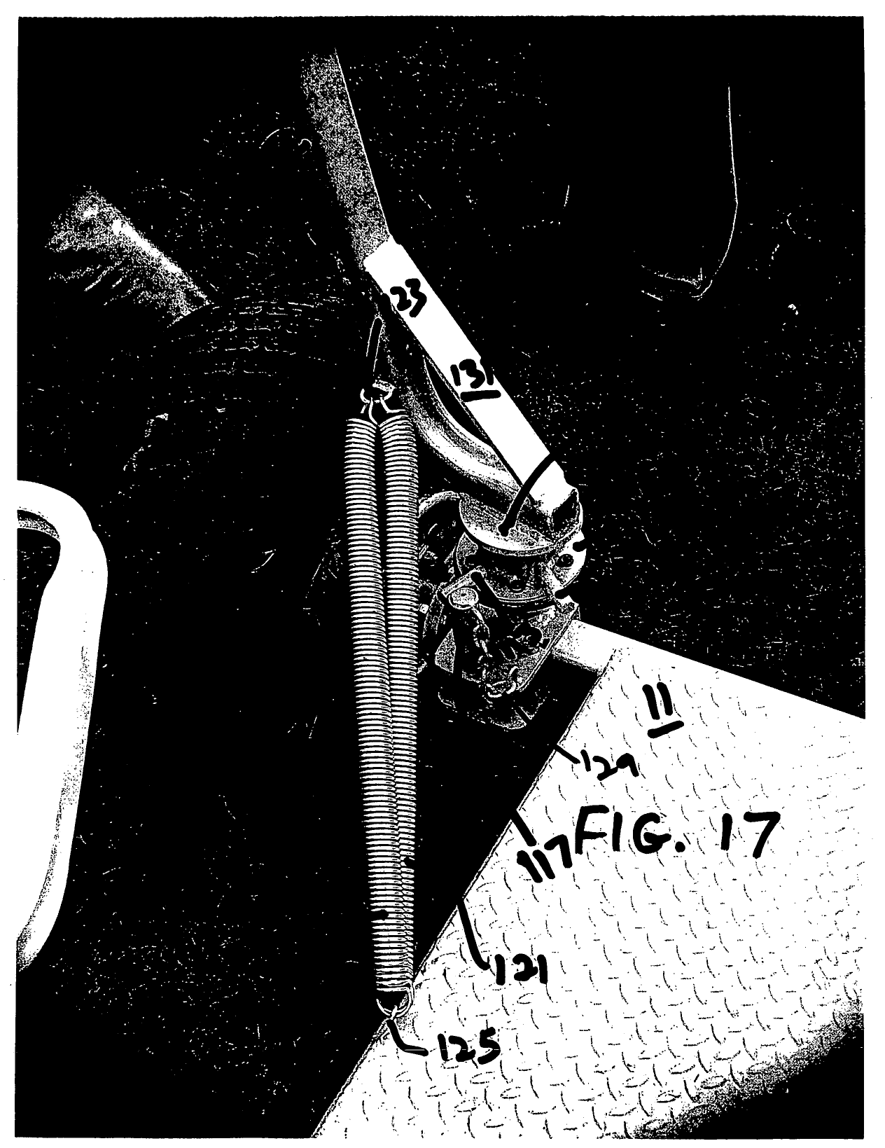

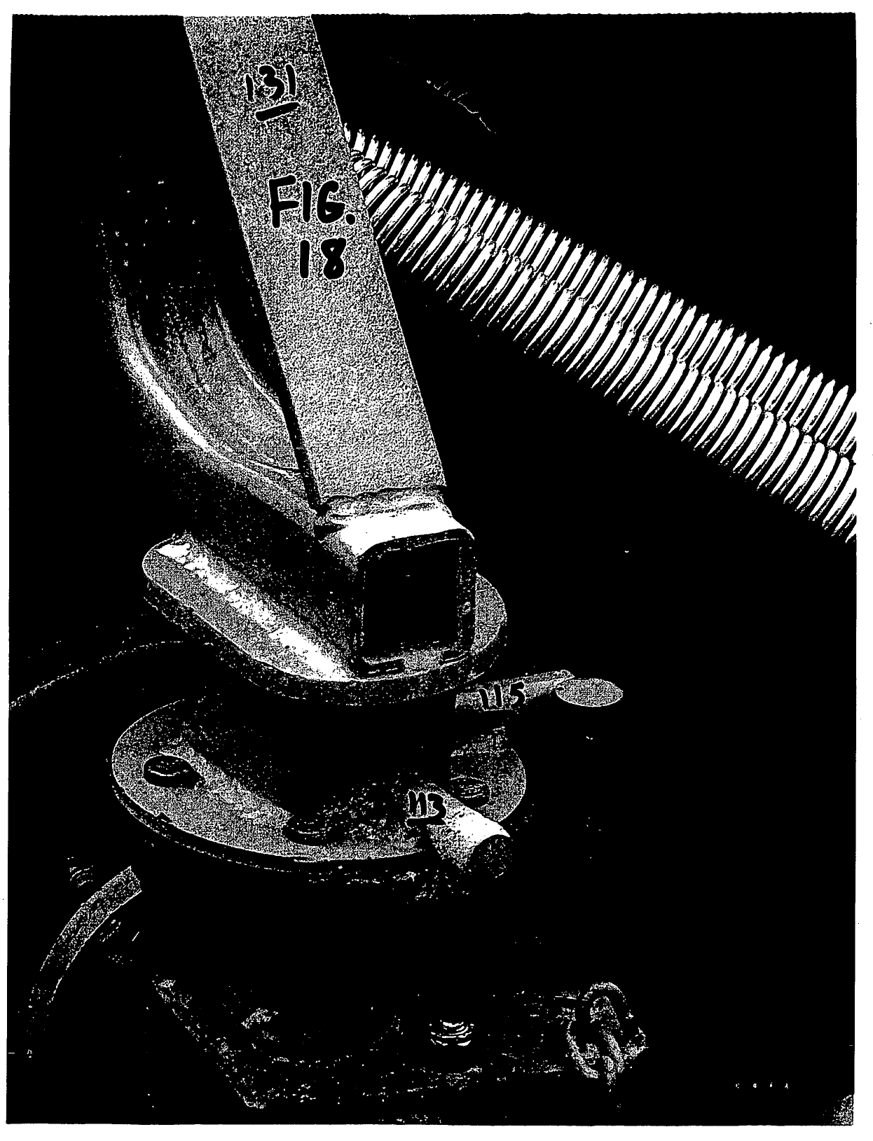

FIG. 16 shows the pad 39 mounted on the arm 33. The chains 47, straps 51, and eyes 49 are clearly visible. FIGS. 17 and 18 show the proximal end of the support 37 which is symmetrical with respect to the proximal end of the support 33. As seen in FIGS. 17 and 18, the support 37 includes a plate 110 including a post (not shown) that fits within a bearing 111 that supports the support 37 for pivoting movements with respect to the frame 11. Limit stops 113 and 115 limit the rotative travel of the support 37 to within about 90°. A post 117 is located between the stops 113 and 115 so that the support 37 can travel therebetween. Tension coil springs 119 and 121 are attached between the support 37 and the frame 11 at eyes 123 and 125 to tend to pivot the support 37 to the position shown, in particular, in FIG. 17 in which the rod 113 engages the stop 117. When a player grasps the pad 41 (FIGS. 2 and 3), he may pivot the support 37 in the clockwise direction in the view of FIG. 17 until the rod 115 engages the post 117 to stop any further pivoting movement. When the pad 41 is released, the tension springs 119 and 121 pivot the support 37 to the position shown in FIG. 17. These features are shown in enlarged detail in FIG. 18. FIG. 18 shows that the limit stop is made up of, in fact, a cap 117 shown in FIG. 17 but not in FIG. 18, which is removable from a post 127 shown in FIG. 18. The purpose for the cap 117 is to reduce the amount of rotative travel of the support 37. In further explanation, the cap 117 has a greater outer diameter than that of the post 127, thus taking up more of a percentage of the distance of travel between the posts 113 and 115. A chain 129 is attached to the cap 117 so that it is not lost if removed from the post 127.

A brace 131 is welded to the support 37 to strengthen it.

The operation of the support 33 holding the pad 39 is symmetric, but otherwise identical to the corresponding operation of the support 37 and the pad 41.

The support 35 for the pad 43 is attached to the frame 11 in a manner similar to that of the supports 37 and 33. However, one difference may be that the angular relationship between the limit stops thereof corresponding to the posts 113 and 115 may subtend a greater angle, up to 180°, to allow the pad 43 to be swung from adjacent the support 37 to adjacent the support 33.

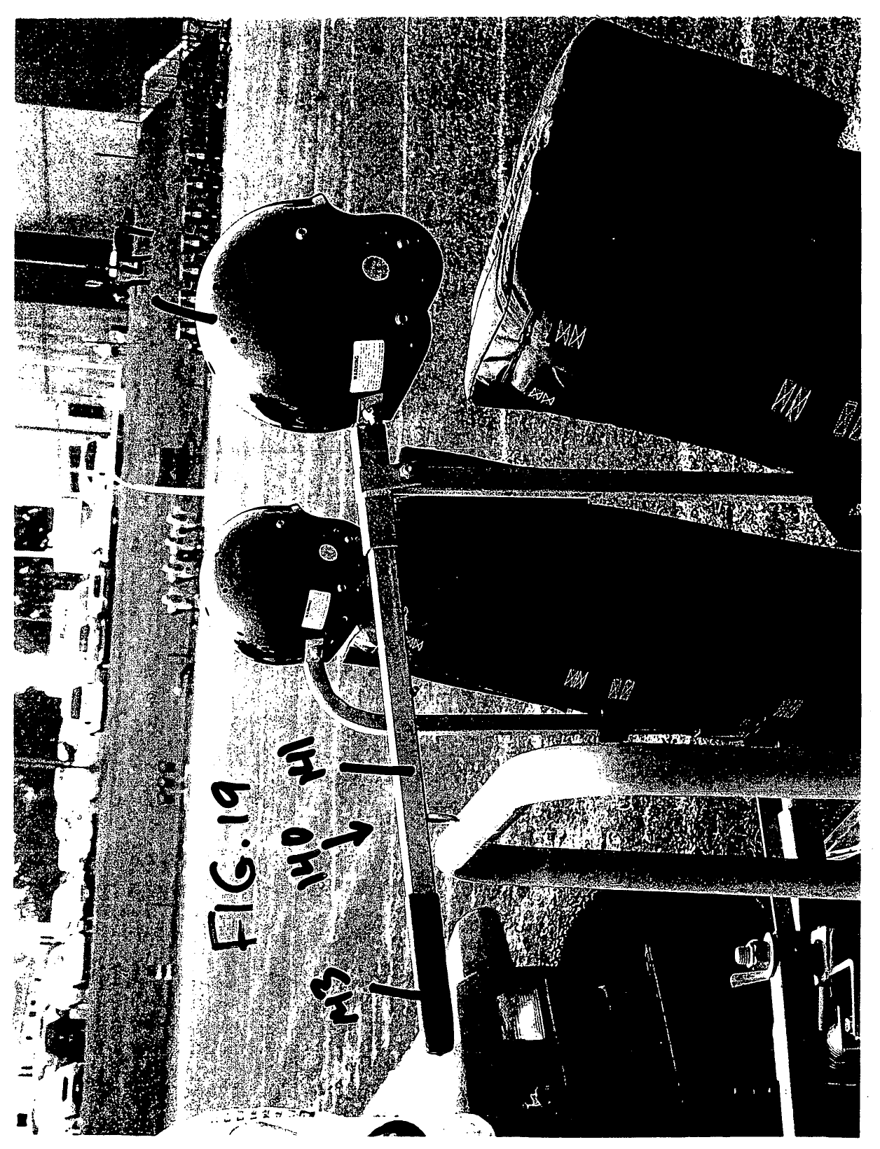

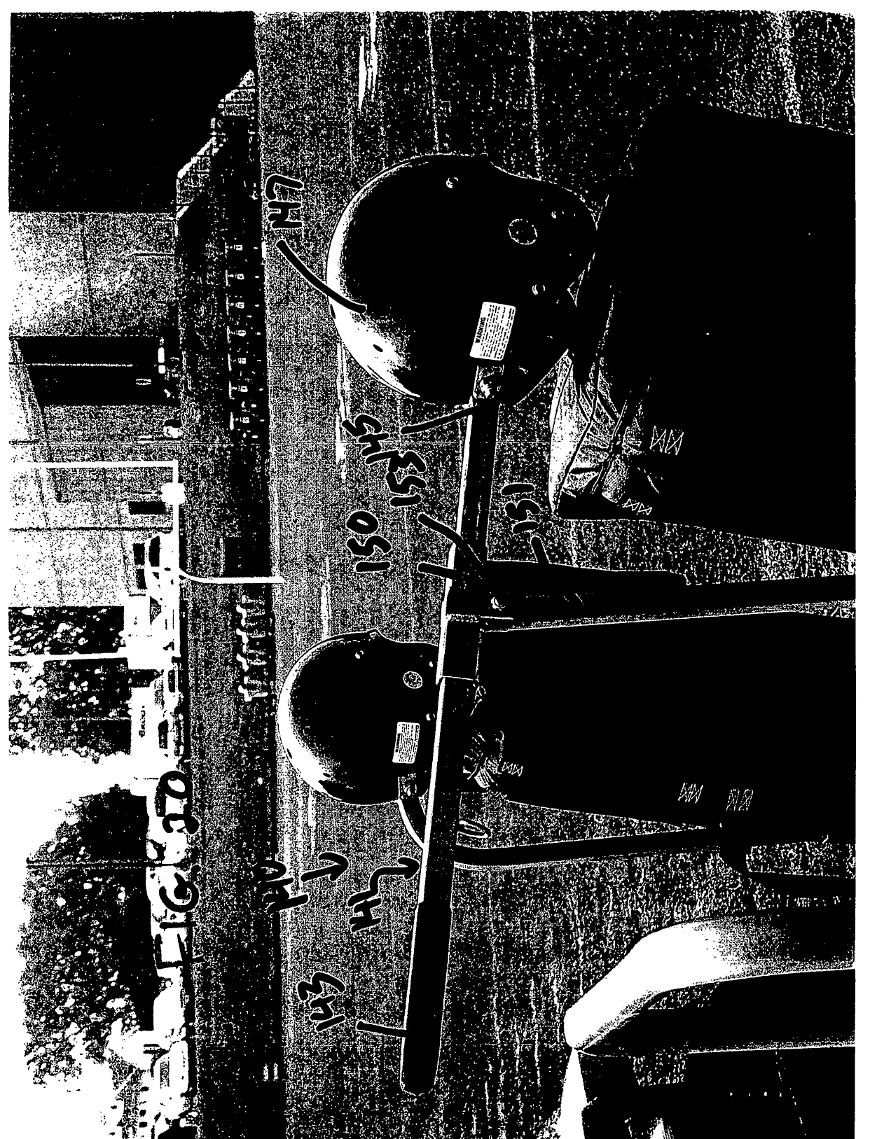

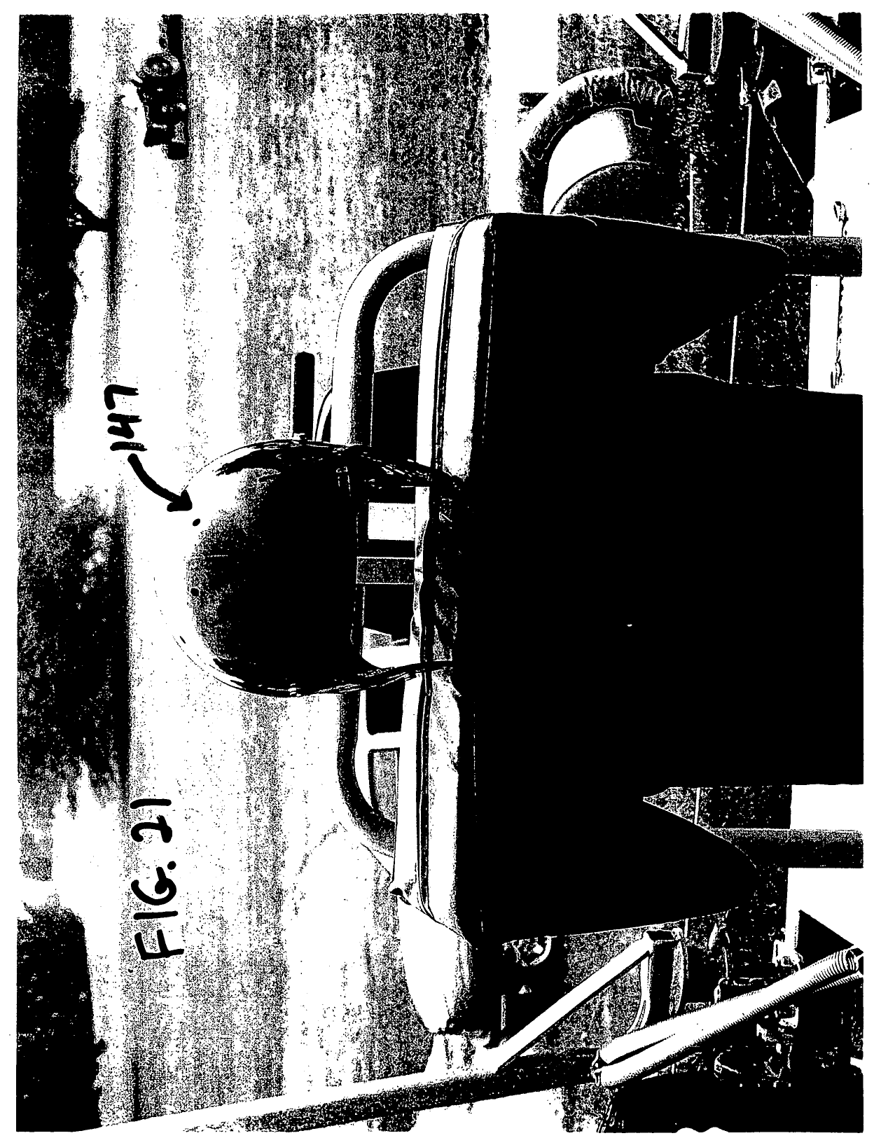

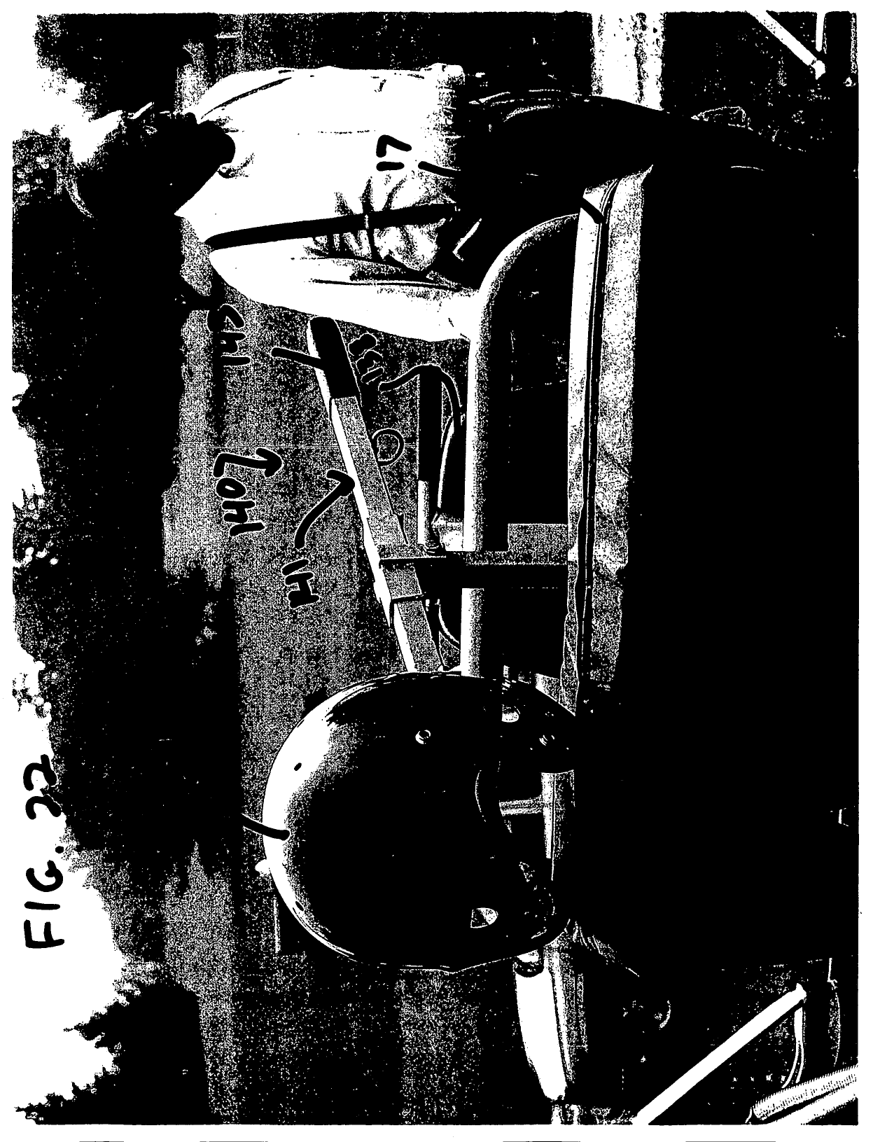

With reference, now, to FIGS. 19-22, an indicator mechanism is generally designated by the reference numeral 140 and comprises a rod seen to include a handle 141, a proximal gripping portion 143, and a distal end 145 to which is attached a football helmet 147. The indicator rod 140 is shown in its retracted centrally aligned position in FIG. 19, and in its extended centrally located position in FIGS. 20 and 21. FIG. 22 shows the indicator rod 140 pivoted so that the helmet 147 is to the left from the front of the device from the perspective shown in FIG. 1. Of course, the indicator rod 140 may also be pivoted so that the helmet 147 rests on the right-hand corner of the pad 17.

With reference back to FIG. 20, it is seen that a sleeve 150 has the rod 140 extending therethrough, and is mounted to a vertical support 151 by a horizontal pivot 153. The support 151 may also rotate with respect to the frame 11 in a manner not shown but well understood by those skilled in the art. Thus, the indicator rod 140 may be moved as should be understood from FIGS. 19-22. The purpose for the indicator 140 is to allow a coach seated on the seat 138 (FIGS. 3 and 22) to grasp the hand grip portion 143 and manipulate the position of the helmet 147 in a manner to cause players using the inventive device 10 to react in an appropriate way. Thus, for example, when the helmet 147 is pivoted to the position shown in FIG. 22, this is done to indicate to the player that he is to block the pad 15, thereby release the further pad 29, block it, and then grab the pad 39 therebehind. Similarly, when the helmet 147 is pivoted to the right in the view of FIG. 22, the coach is instructing the player to hit the pad 19, engage the pad 27, and grab the pad 41. Operation of the indicator 140 is purely manual and at the discretion of the coach. Through use of the inventive device 10, a coach may prepare his players, particularly defensive linemen and linebackers, by simulating actual scenarios that will occur during the playing of a football game where the defensive player will have to defeat the blocks of a plurality of players before being able to approach, hit and tackle a running back, quarterback or receiver.

As such, an invention has been disclosed in terms of a preferred embodiment thereof which fulfills each and every one of the objects of the invention as set forth hereinabove, and provides a new and useful American football defensive player trainer of great novelty and utility.

Of course, various changes, modifications and alterations in the teachings of the present invention may be contemplated by those skilled in the art without departing from the intended spirit and scope thereof.

As such, it is intended that the present invention only be limited by the terms of the appended claims.

1. A football training device, comprising:
a) a frame having a first support supporting a first pad and a second support supporting a second pad;
b) each of said supports supporting its respective pad for movement with respect to said frame;
c) a linkage between said first pad and said second support, said linkage moving between first and second positions responsive to movements of said first pad;
d) movement of said linkage from said first position to said second position causing said second pad to move from a first orientation to a second orientation.

2. The device of claim 1, wherein said first pad is supported on said first support for reciprocating movements with respect to said frame.

3. The device of claim 2, wherein said second pad is supported on said second support for pivoting movements with respect to said frame.

4. The device of claim 1, wherein said linkage comprises:
a) a first arm extending from said first support;
b) a second movable arm mounted on said frame and engageable with said second support, said first arm engaging said second arm remote from said frame;
c) said second arm movable between a first position locking said second support in a locked position and a second position releasing said second support from said locked position.

5. The device of claim 4, wherein said second arm is pivotably mounted on said frame.

6. The device of claim 5, wherein said second support has a slot therein, said slot receiving said second arm therethrough, said second arm having a notch engaging an edge of said slot in said first position and said notch released from said edge in said second position.

7. The device of claim 6, wherein said second pad is supported on said second support for pivoting movements with respect to said frame.

8. The device of claim 7, wherein said second support is biased in a direction of pivoting away from said frame, whereby when said second arm is moved to the second position thereof, releasing said second support, said second support pivots away from said frame.

9. The device of claim 8, wherein said second arm is biased in a direction of said first position.

10. The device of claim 9, wherein said second support is biased by a coil compression spring.

11. The device of claim 10, wherein said second arm is biased toward said first position by a coil tension spring.

12. The device of claim 1, further including a third support supporting a third pad and a fourth support supporting a fourth pad, and a further linkage between said third pad and said fourth support, said further linkage moving between first and second positions responsive to movements of said third pad, movement of said further linkage from said first position to said second position causing said fourth pad to move from a first orientation to a second orientation.

13. The device of claim 12, wherein said first and third pads are mounted on a front edge of said frame.

14. The device of claim 1, further including a third pad hung from a third pivotable support behind said second pad.

15. The device of claim 14, wherein said third support is biased to one position and movable toward a second rotative position.

16. A football training device, comprising:
a) a frame having a first support supporting a first pad, a second support supporting a second pad, a third support supporting a third pad, and a fourth support supporting a fourth pad;
b) each of said supports supporting its respective pad for movement with respect to said frame, said first pad reciprocating with respect to said frame and said second pad pivoting with respect to said frame;
c) a first linkage between said first pad and said second support, said first linkage moving between first and second positions responsive to movements of said first pad;
d) movement of said first linkage from said first position to said second position causing said second pad to move from a first orientation to a second orientation;
e) said third support supporting said third pad for reciprocating with respect to said frame and said fourth support supporting said fourth pad for pivoting with respect to said frame, and a second linkage between said third pad and said fourth support, said second linkage moving between first and second positions responsive to movements of said third pad, movement of said second linkage from said first position to said second position causing said fourth pad to move from a first orientation to a second orientation.

17. The device of claim 16, wherein said first linkage comprises:
a) a first arm extending from said first support;
b) a second movable arm mounted on said frame and engageable with said second support, said first arm engaging said second arm remote from said frame;
c) said second arm movable between a first position locking said second support in a locked position and a second position releasing said second support from said locked position;
d) said second support having a slot therein, said slot receiving said second arm therethrough, said second arm having a notch engaging an edge of said slot in said first position and said notch released from said edge in said second position.

18. The device of claim 17, wherein said second support is biased in a direction of pivoting away from said frame, whereby when said second arm is moved to the second position thereof, releasing said second support, said second support pivots away from said frame.

19. The device of claim 18, wherein said second arm is biased in a direction of said first position by a tension spring, and said second support is biased by a coil compression spring.

20. The device of claim 16, wherein said first and third pads are mounted on a front edge of said frame.
